(19) United States
(12) Patent Application Publication
(10) Pub. No.: US 2015/0342867 A1
(43) Pub. Date: Dec. 3, 2015

(54) Method For Straightening Hair Using Mild Hair Straightening Compositions

(71) Applicant: ELC Management LLC, Melville, NY (US)

(72) Inventors: John Michael Bohen, Lino Lakes, MN (US); Geoffrey Hawkins, Yardley, PA (US); Chad Pavlis, Lino Lakes, MN (US); Karly Rose Slivik, Saint Louis Park, MN (US); Nancy Krueger, Fridley, MN (US); Anita Marie Grahn, Blaine, MN (US)

(21) Appl. No.: 14/724,936

(22) Filed: May 29, 2015

Related U.S. Application Data
(60) Provisional application No. 62/005,123, filed on May 30, 2014.

(51) Int. Cl. A61K 8/89; A61K 8/73; A61Q 5/12

(52) U.S. Cl. CPC A61K 8/89; A61Q 5/12; A61K 8/732; A61K 8/731; A61K 2800/594; A61K 2800/30

(57) ABSTRACT

The present invention relates to a method for straightening hair comprising applying an effective amount of a hair straightening composition to hair, the composition comprising at least one emulsifying silicone elastomer, at least one naturally derived deposition polymer; at least one silicone conditioning agent, and an aqueous carrier, wherein said composition is not rinsed from the hair.

### FIELD OF INVENTION

The present invention relates to a method for straightening human hair. Specifically, the compositions herein are capable of straightening hair using at least one emulsifying silicone elastomer in combination with select conditioning agents, obviating the need for heat and/or reactive hair-straightening chemicals.

### BACKGROUND OF THE INVENTION

Current hair revitalizing and treatment systems involve harsh chemicals, such as oxidizing agents, high concentrations of formaldehyde, and other dangerous chemicals to bind conditioning agents to the hair cuticle. Prior approaches generally require the treatment carriers to first scar the cuticle and then penetrate deeply into the hair shaft, whereupon the reactive agents then substitute some of the conditioning reagents into the hair shaft through the cuticle. Over time, the cortical cells are damaged by these chemicals, potentially destroying the micro-filaments. While these treatments seem to produce desirable results that may gratify some clients, they eventually cause permanent damage to hair. Also, the reactive components of the conditioning treatment may become less efficacious over time, and the consumer's hair will deteriorate, leaving a scarred and damaged hair shaft that requires even further treatment.

Also, when a high concentration of formol is used, the reagents polymerize upon heating the hair with a hot iron, sealing some of the un-reacted agents into the hair shaft for long periods of time. Meanwhile, the hair appears healthy and shiny upon application of these harsh chemicals, but it in fact is slowly being damaged. Precursor agents that existing treatments use, must diffuse deeply into the hair to destroy intrinsic melanin deposits. Repeated use of such harsh chemicals tends to damage the hair significantly. Scalp exposure to the chemicals also may induce allergic reactions in sensitive individuals. Professional hair stylists may even become ill from excessive exposure to the harsh ingredients used by existing hair straightening treatments. One treatment method, for example, relies on lye and other harsh chemicals, while another treatment uses high levels of formaldehyde to achieve straight hair. Alternative treatment methods are needed that produce excellent results without the adverse effects caused by elevated concentrations of harsh chemicals.

Specifically regarding hair straightening techniques, relaxers for hair are known but generally comprise harsh chemicals such as guanidine hydroxide, ammonium thioglycolate, and sodium hydroxide (lye). Therefore, there is a need for hair straightening systems which are free of chemicals such as thioglycolates, and which do not require elevated pH levels.

### SUMMARY OF THE INVENTION

The present invention relates to a method for straightening hair comprising applying an effective amount of a hair straightening composition to hair, the composition comprising at least one emulsifying silicone elastomer, at least one naturally derived deposition polymer; at least one silicone conditioning agent, and an aqueous carrier, wherein said composition is not rinsed from the hair.

### DETAILED DESCRIPTION OF THE INVENTION

While the specification concludes with claims that particularly point out and distinctly claim the invention, it is believed the present invention will be better understood from the following description.

The compositions comprise an emulsifying silicone elastomer, a naturally derived deposition polymer, a silicone conditioning agent, and an aqueous carrier. Each of these essential components, as well as preferred or optional components, is described in detail hereinafter.

All percentages, parts and ratios are based upon the total weight of the compositions, unless otherwise specified. All such weights as they pertain to listed ingredients are based on the active level and, therefore, do not include solvents or by-products that may be included in commercially available materials, unless otherwise specified. The term “weight percent” may be denoted as “wt. %” herein.

All molecular weights as used herein are weight average molecular weights expressed as grams/mole, unless otherwise specified.

The phrase “substantially free of”, as used herein, means that the composition comprises less than 5% by weight of the composition of a stated material.

The term “free of” as used herein, means that the no level of a stated material is intentionally included in the composition. Of course, trace amounts of a material may be present as a result of the manufacturing process.

The term “water-soluble”, as used herein, means that a material is soluble in water in the present invention. In general, the material is soluble at 25° C. at a concentration of at least 0.1% by weight of the water solvent, preferably at least 1%, more preferably at least 5%, most preferably at least 15%.

The term “water-insoluble”, as used herein, means that a compound is not soluble in water in the present composition. Thus, the compound is not miscible with water.

The compositions, described herein, exhibit several important advantages over known compositions for straightening hair. First, the compositions may be free, or substantially free, of reactive hair straightening chemicals, which are known to cause damage to hair over time. Such reactive chemicals include, for example, thiogycolic acid, ammonium thioglycolate, cysteamine, sodium hydroxide, calcium hydroxide, and guanidine hydroxide. The compositions are also preferably free of aldehydes, and specifically formaldehyde or formaldehyde-releasing chemicals. Other chemicals known to reduce sulfur bonds in hair, including mercaptans, sulfonic acids, and sulfites, are also preferably omitted from the compositions herein. Additionally, the compositions do not have an alkaline pH. Rather, the pH of the compositions herein may range from about 3.5 to about 7, more preferably from about 3.5 to about 6, and most preferably from about 4 to about 5. The acidic pH allows for greater formulation flexibility in contrast to alkaline hair straightening compositions. For example, amphoteric surfactants may be utilized at low pH levels to mimic conditioning benefits of cationic surfactants.

### Emulsifying Silicone Elastomer

As used herein, the term “emulsifying silicone elastomer” includes silicone elastomers which comprise at least one hydrophilic chain.

The emulsifying silicone elastomer may be chosen from polyoxyalkylenated silicone elastomers and polyglycerolated silicone elastomers, and mixtures thereof.

The emulsifying silicone elastomer may be present in the compositions at a level of from about 1% to about 20%, more preferably from about 2% to about 15%, and most preferably from about 3 to about 9%.

Polyoxyalkylenated Silicone Elastomers

The polyoxyalkylenated silicone elastomer is a crosslinked organopolysiloxane that can be obtained by the crosslinking addition reaction of diorganopolysiloxane, containing at least one hydrogen bonded to silicon, and of a polyoxyalkylene having at least two ethylenically unsaturated groups. Such reactions are discussed in detail in U.S. Pat. Nos. 5,236,986 and 5,412,004.

Exemplary polyoxyalkylenated elastomers are described in U.S. Pat. No. 5,236,986, U.S. Pat. No. 5,412,004, U.S. Pat. No. 5,837,793 and U.S. Pat. No. 5,811,487.

Specific exemplary polyoxyalkylenated silicone elastomers include those sold under the names KSG-21, KSG-20, KSG-30, KSG-31, KSG-32, KSG-33, KSG-210, KSG-310, KSG-320, KSG-330, KSG-340 and X-226146 by Shin-Etsu, and DC9010 and DC9011 by Dow Corning.

According to one preferred embodiment, the polyoxyalkylenated silicone elastomer sold under the name KSG-210 by Shin-Etsu is utilized in the compositions herein.

Polyglycerolated Silicone Elastomers

The emulsifying silicone elastomer may also be chosen from polyglycerolated silicone elastomers.

The polyglycerolated silicone elastomer is a crosslinked elastomeric organopolysiloxane that can be obtained by a crosslinking addition reaction of diorganopolysiloxane containing at least one hydrogen bonded to silicon and of polyglycerolated compounds having ethylenically unsaturated groups, in particular in the presence of a platinum catalyst.

Preferably, the crosslinked elastomeric organopolysiloxane is obtained by crosslinking addition reaction (A) of diorganopolysiloxane containing at least two hydrogens each bonded to a silicon, and (B) of glycerolated compounds having at least two ethylenically unsaturated groups, in particular in the presence (C) of a platinum catalyst.

In particular, the organopolysiloxane can be obtained by reaction of a polyglycerolated compound containing dimethylvinylsiloxy end groups and of methylhydrogenopolysiloxane containing trimethylsiloxy end groups, in the presence of a platinum catalyst.

### Naturally Derived Deposition Polymer

The compositions comprise at least one naturally derived cationic polymer. The term, “naturally derived cationic polymer” as used herein, refers to cationic polymers which are obtained from natural sources. The natural sources may be selected from celluloses, starches, galactomannans and other sources found in nature. The naturally derived cationic polymer has a molecular weight from about 1,000 to about 10,000,000, and a cationic charge density at least about 3.0 meq/g, more preferably at least about 3.2 meq/g. Preferably the cationic charge density is also less than about 7 meq/g. The naturally derived polymers are present in an amount of at least 0.05 wt. % of the composition. Preferably, the polymers are present at a range of from about 0.05% to about 10%, and more preferably from about 0.05% to about 5%, by weight of the composition.

The naturally derived cationic polymers are generally water soluble and aid in deposition of the silicone conditioning agents described herein. Such deposition enhancement results in improved hair feel, wet conditioning, shine and other appreciable benefits.

Cellulose or Guar Cationic Deposition Polymers

The compositions may include cellulose or guar cationic deposition polymers. Such cellulose or guar deposition polymers have a charge density from about 3 meq/g to about 4.0 meq/g at the pH of intended use of the composition, which pH will generally range from about pH 3 to about pH 9, preferably between about pH 4 and about pH 8. The pH of the compositions are measured neat.

Suitable cellulose cationic polymers include those which conform to the following formula:

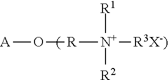

wherein A is an anhydroglucose residual group, such as a cellulose anhydroglucose residual; R is an alkylene oxyalkylene, polyoxyalkylene, or hydroxyalkylene group, or combination thereof, R¹, R², and R³ independently are alkyl, aryl, alkylaryl, arylalkyl, alkoxyalkyl, or alkoxyaryl groups, each group containing up to about 18 carbon atoms, and the total number of carbon atoms for each cationic moiety (i.e., the sum of carbon atoms in R¹, R² and R³) preferably being about 20 or less; and X is an anionic counterion. Non-limiting examples of such counterions include halides (e.g., chlorine, fluorine, bromine, iodine), sulfate and methylsulfate. The degree of cationic substitution in these polysaccharide polymers is typically from about 0.01 to about 1 cationic groups per anhydroglucose unit.

In one embodiment of the invention, the cellulose polymers are salts of hydroxyethyl cellulose reacted with trimethyl ammonium substituted epoxide, referred to in the industry (CTFA) as Polyquatemium 10 and available from Amerchol Corp. (Edison, N.J., USA).

Other suitable cationic deposition polymers include cationic guar gum derivatives, such as guar hydroxypropyltrimonium chloride, specific examples of which include the Jaguar series (preferably Jaguar® C-17R) commercially available from RhoneRhodia.

Cationically Modified Starch Polymer

The compositions may also comprise a water-soluble cationically modified starch polymer. As used herein, the term “cationically modified starch” refers to a starch to which a cationic group is added prior to degradation of the starch to a smaller molecular weight, or wherein a cationic group is added after modification of the starch to achieve a desired molecular weight. The definition of the term “cationically modified starch” also includes amphoterically modified starch. The term “amphoterically modified starch” refers to a starch hydrolysate to which a cationic group and an anionic group are added.

The cationically modified starch polymers disclosed herein have a percent of bound nitrogen of from about 0.5% to about 4%. The cationically modified starch polymers also have a molecular weight of from about 50,000 to about 10,000,000.

The cationically modified starch polymers have a charge density at least about 3.0 meq/g. The chemical modification to obtain such a charge density includes, but is not limited to, the addition of amino and/or ammonium groups into the starch molecules. Non-limiting examples of these ammonium groups may include substituents such as hydroxypropyl trimmonium chloride, trimethylhydroxypropyl ammonium chloride, dimethylstearylhydroxypropyl ammonium chloride, and dimethyldodecylhydroxypropyl ammonium chloride. See Solarek, D. B., Cationic Starches in Modified Starches: Properties and Uses, Wurzburg, O. B., Ed., CRC Press, Inc., Boca Raton, Fla. 1986, pp 113-125. The cationic groups may be added to the starch prior to degradation to a smaller molecular weight or the cationic groups may be added after such modification.

As used herein, the “degree of substitution” of the cationically modified starch polymers is an average measure of the number of hydroxyl groups on each anhydroglucose unit which is derivatized by substituent groups. Since each anhydroglucose unit has three potential hydroxyl groups available for substitution, the maximum possible degree of substitution is 3. The degree of substitution is expressed as the number of moles of substituent groups per mole of anhydroglucose unit, on a molar average basis. The degree of substitution may be determined using proton nuclear magnetic resonance spectroscopy (“¹H NMR”) methods well known in the art. Suitable ¹H NMR techniques include those described in “Observation on NMR Spectra of Starches in Dimethyl Sulfoxide, Iodine-Complexing, and Solvating in Water-Dimethyl Sulfoxide”, Qin-Ji Peng and Arthur S. Perlin, Carbohydrate Research, 160 (1987), 57-72; and “An Approach to the Structural Analysis of Oligosaccharides by NMR Spectroscopy”, J. Howard Bradbury and J. Grant Collins, Carbohydrate Research, 71, (1979), 15-25.

The cationically modified starch polymer may comprise maltodextrin. Thus, in one embodiment, the cationically modified starch polymers may be further characterized by a Dextrose Equivalance (“DE”) value of less than about 35, and more preferably from about 1 to about 20. The DE value is a measure of the reducing equivalence of the hydrolyzed starch referenced to dextrose and expressed as a percent (on dry basis). Starch completely hydrolyzed to dextrose has a DE value of 100, and unhydrolyzed starch has a DE value of 0. A suitable assay for DE value includes one described in “Dextrose Equivalent”, Standard Analytical Methods of the Member Companies of the Corn Industries Research Foundation, 1st ed., Method E-26. Additionally, the cationically modified starch polymers may comprise a dextrin. Dextrin is typically a pyrolysis product of starch with a wide range of molecular weights.

The source of starch before chemical modification can be chosen from a variety of sources such as tubers, legumes, cereal, and grains. Non-limiting examples of this source starch may include corn starch, wheat starch, rice starch, waxy corn starch, oat starch, cassia starch, waxy barley, waxy rice starch, glutenous rice starch, sweet rice starch, amioca, potato starch, tapioca starch, oat starch, sago starch, sweet rice, or mixtures thereof. Corn starch and tapioca starch are preferred.

In one embodiment, cationically modified starch polymers are selected from degraded cationic maize starch, cationic tapioca, cationic potato starch, and mixtures thereof. In another embodiment, cationically modified starch polymers are cationic corn starch. The starch, prior to degradation or after modification to a smaller molecular weight, may comprise one or more additional modifications. For example, these modifications may include cross-linking, stabilization reactions, phosphorylations, and hydrolyzations. Stabilization reactions may include alkylation and esterification.

The cationically modified starch polymers may be incorporated into the composition in the form of hydrolyzed starch (e.g., acid, enzyme, or alkaline degradation), oxidized starch (e.g., peroxide, peracid, hypochlorite, alkaline, or any other oxidizing agent), physically/mechanically degraded starch (e.g., via the thermo-mechanical energy input of the processing equipment), or combinations thereof.

Also suitable for use in the present invention is nonionic modified starch that could be further derivatized to a cationically modified starch as is known in the art. Other suitable modified starch starting materials may be quaternized, as is known in the art, to produce the cationically modified starch polymer suitable for use in the invention.

Starch Degradation Procedure

In one embodiment, a starch slurry is prepared by mixing granular starch in water. The temperature is raised to about 35° C. An aqueous solution of potassium permanganate is then added at a concentration of about 50 ppm based on starch. The pH is raised to about 11.5 with sodium hydroxide and the slurry is stirred sufficiently to prevent settling of the starch. Then, about a 30% solution of hydrogen peroxide diluted in water is added to a level of about 1% of peroxide based on starch. The pH of about 11.5 is then restored by adding additional sodium hydroxide. The reaction is completed over about a 1 to about 20 hour period. The mixture is then neutralized with dilute hydrochloric acid. The degraded starch is recovered by filtration followed by washing and drying.

Galactomannan Polymer Derivative

The compositions may comprise a galactomannan polymer derivative having a mannose to galactose ratio of greater than 2:1 on a monomer to monomer basis, the galactomannan polymer derivative is selected from the group consisting of a cationic galactomannan polymer derivative and an amphoteric galactomannan polymer derivative having a net positive charge. The term “galactomannan polymer derivative”, means a compound obtained from a galactomannan polymer (ie. a galactomannan gum). As used herein, the term “cationic galactomannan” refers to a galactomannan polymer to which a cationic group is added. The term “amphoteric galactomannan” refers to a galactomannan polymer to which a cationic group and an anionic group are added such that the polymer has a net positive charge.

In one embodiment, the galactomannan is a non-guar galactomannan. The gum for use in preparing the non-guar galactomannan polymer derivatives is typically obtained as naturally occurring material such as seeds or beans from plants. Examples of various non-guar galactomannan polymers include but are not limited to Tara gum (3 parts mannose/1 part galactose), Locust bean or Carob (4 parts mannose/1 part galactose), and Cassia gum (5 parts mannose/1 part galactose). Herein, the term “non-guar galactomannan polymer derivatives” refers to cationic polymers which are chemically modified from a non-guar galactomanan polymer. A preferred non-guar galactomannan polymer derivative is cationic cassia, which is sold under the trade name, Cassia EX-906, and is commercially available from Noveon Inc.

The galactomannan polymer derivatives have a molecular weight of from about 1,000 to about 10,000,000. In one embodiment, the galactomannan polymer derivatives have a molecular weight of from about 5,000 to about 3,000,000. As used herein, the term “molecular weight” refers to the weight average molecular weight. The weight average molecular weight may be measured by gel permeation chromatography. Exemplary galactomannan polymer derivatives are described in U.S. Patent Publication No. 2006/0099167A 1 to Staudigel et al.

### Silicone Conditioning Agent

The compositions also include at least one nonvolatile soluble or insoluble silicone conditioning agent. By soluble what is meant is that the silicone conditioning agent is miscible with the aqueous carrier of the composition so as to form part of the same phase. By insoluble what is meant is that the silicone forms a separate, discontinuous phase from the aqueous carrier, such as in the form of an emulsion or a suspension of droplets of the silicone.

The silicone hair conditioning agent may be used in the compositions at levels of from about 0.05% to about 20% by weight of the composition, preferably from about 0.1% to about 10%, more preferably from about 0.5% to about 5%, most preferably from about 0.5% to about 3%.

Soluble silicones include silicone copolyols, such as dimethicone copolyols, e.g. polyether siloxane-modified polymers, such as polypropylene oxide, polyethylene oxide modified polydimethylsiloxane, wherein the level of ethylene and/or propylene oxide sufficient to allow solubility in the composition.

Insoluble silicones are also useful in the present invention. The insoluble silicone hair conditioning agent for use herein will preferably have viscosity of from about 1,000 to about 2,000,000 centistokes at 25° C., more preferably from about 10,000 to about 1,800,000, even more preferably from about 100,000 to about 1,500,000. The viscosity can be measured by means of a glass capillary viscometer as set forth in Dow Corning Corporate Test Method CTM0004, Jul. 20, 1970.

In one embodiment, the compositions include at least a first and second silicone conditioning agent. The first silicone conditioning agent has a relatively low viscosity of from about 1 to about 100, more preferably from about 3 to about 50, and most preferably from about 5 to about 10 centistokes at 25° C., and the second silicone conditioning agent has a viscosity of from about 1,000 to about 2,000,000, more preferably from about 10,000 to about 1,000,000, and most preferably from about 50,000 to 200,000 centistokes at 25° C. It is believed that providing at least two silicone conditioning agents, according to the first and second silicone conditioning agents discussed herein, provides improved spreadability, slip, and decreased styling time to achieve a straight, smooth style. Suitable insoluble, nonvolatile silicone fluids include polyalkyl siloxanes, polyaryl siloxanes, polyalkylaryl siloxanes, polyether siloxane copolymers, and mixtures thereof. Other insoluble, nonvolatile silicone fluids having hair conditioning properties can also be used. The term “nonvolatile” as used herein shall mean that the silicone has a boiling point of at least about 260° C., preferably at least about 275° C., more preferably at least about 300° C. Such materials exhibit very low or no significant vapor pressure at ambient conditions. The term “silicone fluid” shall mean flowable silicone materials having a viscosity of less than 1,000,000 centistokes at 25° C. Generally, the viscosity of the fluid will be between about 5 and 1,000,000 centistokes at 25° C., preferably between about 10 and about 300,000 centistokes.

Silicone fluids hereof also include polyalkyl or polyaryl siloxanes with the following structure:

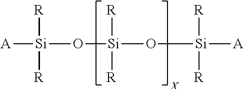

wherein R is alkyl or aryl, and x is an integer from about 7 to about 8,000 may be used. “A” represents groups which block the ends of the silicone chains.

The alkyl or aryl groups substituted on the siloxane chain (R) or at the ends of the siloxane chains (A) may have any structure as long as the resulting silicones remain fluid at room temperature, are hydrophobic, are neither irritating, toxic nor otherwise harmful when applied to the hair, are compatible with the other components of the composition, are chemically stable under normal use and storage conditions, and are capable of being deposited on and of conditioning hair.

Suitable A groups include methyl, methoxy, ethoxy, propoxy, and aryloxy. The two R groups on the silicone atom may represent the same group or different groups. Preferably, the two R groups represent the same group. Suitable R groups include methyl, ethyl, propyl, phenyl, methylphenyl and phenylmethyl. The preferred silicones are polydimethyl siloxane, polydiethylsiloxane, and polymethylphenylsiloxane. Polydimethylsiloxane is especially preferred.

The nonvolatile polyalkylsiloxane fluids that may be used include, for example, polydimethylsiloxanes. These siloxanes are available, for example, from the General Electric Company in their ViscasilR and SF 96 series, and from Dow Corning in their Dow Corning 200 series.

The polyalkylaryl siloxane fluids that may be used, also include, for example, polymethylphenylsiloxanes. These siloxanes are available, for example, from the General Electric Company as SF 1075 methyl phenyl fluid or from Dow Corning as 556 Cosmetic Grade Fluid.

Especially preferred, for enhancing the shine characteristics of hair, are highly arylated silicones, such as highly phenylated polyethyl silicone having refractive indices of about 1.46 or higher, especially about 1.52 or higher. When these high refractive index silicones are used, they should be mixed with a spreading agent, such as a surfactant or a silicone resin, as described below to decrease the surface tension and enhance the film forming ability of the material.

The polyether siloxane copolymers that may be used include, for example, a polypropylene oxide modified polydimethylsiloxane (e.g., Dow Corning DC-1248) although ethylene oxide or mixtures of ethylene oxide and propylene oxide may also be used. The ethylene oxide and polypropylene oxide level should be sufficiently low to prevent solubility in the composition hereof.

References disclosing suitable silicone fluids include U.S. Pat. No. 2,826,551, Geen; U.S. Pat. No. 3,964,500, Drakoff, issued Jun. 22, 1976; U.S. Pat. No. 4,364,837, Pader; and British Patent 849,433, Woolston. Also useful are Silicon Compounds distributed by Petrarch Systems, Inc., 1984. This reference provides an extensive (though not exclusive) listing of suitable silicone fluids.

Another silicone hair conditioning material that can be especially useful in the silicone conditioning agents is insoluble silicone gum. The term “silicone gum”, as used herein, means polyorganosiloxane materials having a viscosity at 25° C. of greater than or equal to 1,000,000 centistokes. Silicone gums are described by Petrarch and others including U.S. Pat. No. 4,152,416, Spitzer et al., issued May 1, 1979 and Noll, Walter, Chemistry and Technology of Silicones, New York: Academic Press 1968. Also describing silicone gums are General Electric Silicone Rubber Product Data Sheets SE 30, SE 33, SE 54 and SE 76. The “silicone gums” will typically have a mass molecular weight in excess of about 200,000, generally between about 200,000 and about 1,000,000. Specific examples include polydimethylsiloxane, (polydimethylsiloxane) (methylvinylsiloxane) copolymer, poly(di-methylsiloxane) (diphenyl siloxane)(methylvinylsiloxane) copolymer and mixtures thereof.

In one embodiment, the silicone hair conditioning agent comprises a mixture of a polydimethylsiloxane gum, having a viscosity greater than about 1,000,000 centistokes and polydimethylsiloxane fluid having a viscosity of from about 10 centistokes to about 100,000 centistokes, wherein the ratio of gum to fluid is from about 30:70 to about 70:30, preferably from about 40:60 to about 60:40.

An optional ingredient that can be included in the silicone conditioning agent is silicone resin. Silicone resins are highly crosslinked polymeric siloxane systems. The cross-linking is introduced through the incorporation of trifunctional and tetrafunctional silanes with monofunctional or difunctional, or both, silanes during manufacture of the silicone resin. As is well understood in the art, the degree of crosslinking that is required in order to result in a silicone resin will vary according to the specific silane units incorporated into the silicone resin. In general, silicone materials which have a sufficient level of trifunctional and tetrafunctional siloxane monomer units (and hence, a sufficient level of crosslinking) such that they dry down to a rigid, or hard, film are considered to be silicone resins. The ratio of oxygen atoms to silicon atoms is indicative of the level of crosslinking in a particular silicone material. Silicone materials which have at least about 1.1 oxygen atoms per silicon atom will generally be silicone resins herein. Preferably, the ratio of oxygen:silicon atoms is at least about 1.2:1.0. Silanes used in the manufacture of silicone resins include monomethyl-, dimethyl-, trimethyl-, monophenyl-, diphenyl-, methylphenyl-, monovinyl-, and methylvinyl-chlorosilanes, and tetrachlorosilane, with the methyl-substituted silanes being most commonly utilized. Preferred resins are offered by General Electric as GE SS4230 and SS4267. Commercially available silicone resins will generally be supplied in a dissolved form in a low viscosity volatile or nonvolatile silicone fluid. The silicone resins for use herein should be supplied and incorporated into the present compositions in such dissolved form, as will be readily apparent to those skilled in the art.

Silicone resins can enhance deposition of silicone on the hair and can enhance the glossiness of hair with high refractive index volumes.

Background material on silicones including sections discussing silicone fluids, gums, and resins, as well as manufacture of silicones, can be found in Encyclopedia of Polymer Science and Engineering, Volume 15, Second Edition, pp 204-308, John Wiley & Sons, Inc., 1989.

Silicone materials and silicone resins in particular, can conveniently be identified according to a shorthand nomenclature system well known to those skilled in the art as “MDTQ” nomenclature. Under this system, the silicone is described according to presence of various siloxane monomer units which make up the silicone. Briefly, the symbol M denotes the monofunctional unit (CH₃)₃SiO)_{0.5}; D denotes the difunctional unit (CH₃)₂SiO; T denotes the trifunctional unit (CH₃)SiO_{1.5}; and Q denotes the quadri- or tetra-functional unit SiO₂. Primes of the unit symbols, e.g., M′, D′, T′, and Q′ denote substituents other than methyl, and must be specifically defined for each occurrence. Typical alternate substituents include groups such as vinyl, phenyls, amines, hydroxyls, etc. The molar ratios of the various units, either in terms of subscripts to the symbols indicating the total number of each type of unit in the silicone (or an average thereof) or as specifically indicated ratios in combination with molecular weight complete the description of the silicone material under the MDTQ system. Higher relative molar amounts of T, Q, T′ and/or Q′ to D, D′, M and/or or M′ in a silicone resin is indicative of higher levels of crosslinking. As discussed before, however, the overall level of crosslinking can also be indicated by the oxygen to silicon ratio.

The silicone resins for use herein which are preferred are MQ, MT, MTQ, MQ and MDTQ resins. Thus, the preferred silicone substituent is methyl. Especially preferred are MQ resins wherein the M:Q ratio is from about 0.5:1.0 to about 1.5:1.0 and the average molecular weight of the resin is from about 1000 to about 10,000.

### Additional Conditioning Agents

The compositions also comprise one or more additional conditioning agents, such as those selected from the group consisting of cationic surfactants, cationic polymers, nonvolatile silicones (including soluble and insoluble silicones), nonvolatile hydrocarbons, saturated C₁₄ to C₂₂ straight chain fatty alcohols, nonvolatile hydrocarbon esters, and mixtures thereof. Preferred conditioning agents are cationic surfactants, cationic polymers, saturated C₁₄ to C₂₂ straight chain fatty alcohols, and quarternary ammonium salts. The components hereof can comprise from about 0.1% to about 99%, more preferably from about 0.5% to about 90%, of conditioning agents. However, in the presence of an aqueous carrier, the conditioning agents preferably comprise from about 0.1% to about 90%, more preferably from about 0.5 to about 60% and most preferably from about 1% to about 50% by weight of the composition.

Cationic Surfactants

Cationic surfactants, useful in the present compositions, contain amino or quaternary ammonium moieties. The cationic surfactant will preferably, though not necessarily, be insoluble in the compositions hereof. Cationic surfactants among those useful herein are disclosed in the following documents: M.C. Publishing Co., McCutcheon's, Detergents & Emulsifiers, (North American edition 1979); Schwartz, et al., Surface Active Agents, Their Chemistry and Technology, New York: Interscience Publishers, 1949; U.S. Pat. No. 3,155,591, Hilfer, issued Nov. 3, 1964; U.S. Pat. No. 3,929,678, Laughlin et al., issued Dec. 30, 1975; U.S. Pat. No. 3,959,461, Bailey et al., issued May 25, 1976; and U.S. Pat. No. 4,387,090, Bolich, Jr., issued Jun. 7, 1983.

Among the quaternary ammonium-containing cationic surfactant materials useful herein are those of the general formula:

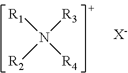

wherein R₁-R₄ are independently an aliphatic group of from about 1 to about 22 carbon atoms or an aromatic, alkoxy, polyoxyalkylene, alkylamido, hydroxyalkyl, aryl or alkylaryl group having from about 1 to about 22 carbon atoms; and X is a salt-forming anion such as those selected from halogen, (e.g. chloride, bromide), acetate, citrate, lactate, glycolate, phosphate nitrate, sulfate, and alkylsulfate radicals. The aliphatic groups may contain, in addition to carbon and hydrogen atoms, ether linkages, and other groups such as amino groups. The longer chain aliphatic groups, e.g., those of about 12 carbons, or higher, can be saturated or unsaturated. Especially preferred are di-long chain (e.g., di C₁₂-C₂₂, preferably C₁₆-C₁₈, aliphatic, preferably alkyl). di-short chain (e.g., C₁-C₃ alkyl, preferably C₁-C₂ alkyl) quaternary ammonium salts,

Salts of primary, secondary and tertiary fatty amines are also suitable cationic surfactant materials. The alkyl groups of such amines preferably have from about 12 to about 22 carbon atoms, and may be substituted or unsubstituted. Such amines, useful herein, include stearamido propyl dimethyl amine, diethyl amino ethyl stearamide, dimethyl stearamine, dimethyl soyamine, soyamine, myristyl amine, tridecyl amine, ethyl stearylamine, N-tallowpropane diamine, ethoxylated (with 5 moles of ethylene oxide) stearylamine, dihydroxy ethyl stearylamine, and arachidylbehenylamine. Suitable amine salts include the halogen, acetate, phosphate, nitrate, citrate, lactate, and alkyl sulfate salts. Such salts include stearylamine hydrochloride, soyamine chloride, stearylamine formate, N-tallowpropane diamine dichloride and stearamidopropyl dimethylamine citrate. Cationic amine surfactants included among those useful in the present invention are disclosed in U.S. Pat. No. 4,275,055, Nachtigal, et al., issued Jun. 23, 1981.

Cationic surfactants may preferably be utilized at levels of from about 0.1% to about 10%, more preferably from about 0.25% to about 5%, most preferably from about 0.5% to about 2%, by weight of the composition.

Cationic Polymer Conditioning Agent

In addition to the naturally derived cationic starch polymers herein, the compositions may also comprise one or more additional cationic polymer conditioning agents. The cationic polymer conditioning agents will preferably be water soluble. Cationic polymers are typically used in the same ranges as disclosed above for cationic surfactants.

The cationic polymers hereof will generally have a weight average molecular weight which is at least about 5,000, typically at least about 10,000, and is less than about 10 million. Preferably, the molecular weight is from about 100,000 to about 2 million. The cationic polymers will generally have cationic nitrogen-containing moieties such as quaternary ammonium or cationic amino moieties, and mixtures thereof.

The cationic charge density is preferably at least about 0.1 meq/g, more preferably at least about 1.5 meq/g, even more preferably at least abut 1.1 meq/g, most preferably at least about 1.2 meq/g. The “cationic charge density” of a polymer, as that term is used herein, refers to the ratio of the number of positive charges on a monomeric unit of which the polymer is comprised to the molecular weight of said monomeric unit. The cationic charge density multiplied by the polymer molecular weight determines the number of positively charged sites on a given polymer chain. The average molecular weight of such suitable cationic polymers will generally be between about 10,000 and 10 million, preferably between about 50,000 and about 5 million, more preferably between about 100,000 and about 3 million. Those skilled in the art will recognize that the charge density of amino-containing polymers may vary depending upon pH and the isoelectric point of the amino groups. The charge density should be within the above limits at the pH of intended use.

Any anionic counterions can be utilized for the cationic polymers so long as the water solubility criteria is met. Suitable counterions include halides (e.g., Cl, Br, I, or F, preferably Cl, Br, or I), sulfate, and methylsulfate. Others can also be used, as this list is not exclusive.

The cationic nitrogen-containing moiety will be present generally as a substituent, on a fraction of the total monomer units of the cationic hair conditioning polymers. Thus, the cationic polymer can comprise copolymers, terpolymers, etc. of quaternary ammonium or cationic amine-substituted monomer units and other non-cationic units referred to herein as spacer monomer units. Such polymers are known in the art, and a variety can be found in the CTFA Cosmetic Ingredient Dictionary, 3rd edition, edited by Estrin, Crosley, and Haynes, (The Cosmetic, Toiletry, and Fragrance Association, Inc., Washington, D.C., 1982).

Suitable cationic polymers include, for example, copolymers of vinyl monomers having cationic amine or quaternary ammonium functionalities with water soluble spacer monomers such as acrylamide, methacrylamide, alkyl and dialkyl acrylamides, alkyl and dialkyl methacrylamides, alkyl acrylate, alkyl methacrylate, vinyl caprolactone, and vinyl pyrrolidone. The alkyl and dialkyl substituted monomers preferably have C₁-C₇ alkyl groups, more preferably C₁-C₃ alkyl groups. Other suitable spacer monomers include vinyl esters, vinyl alcohol (made by hydrolysis of polyvinyl acetate), maleic anhydride, propylene glycol, and ethylene glycol.

The cationic amines can be primary, secondary, or tertiary amines, depending upon the particular species and the pH of the composition. In general, secondary and tertiary amines, especially tertiary amines, are preferred.

The cationic polymers hereof can comprise mixtures of monomer units derived from amine- and/or quaternary ammonium-substituted monomer and/or compatible spacer monomers.

Suitable cationic hair conditioning polymers include, for example: copolymers of 1-vinyl-2-pyrrolidone and 1-vinyl-3-methylimidazolium salt (e.g., chloride salt) (referred to in the industry by the Cosmetic, Toiletry, and Fragrance Association, “CTFA”, as Polyquaternium-16), such as those commercially available from BASF Wyandotte Corp. (Parsippany, N.J., USA) under the LUVIQUAT tradename (e.g., LUVIQUAT FC 370); co-polymers of 1-vinyl-2-pyrrolidone and dimethylaminoethyl methacrylate (referred to in the industry by CTFA as Polyquaternium-11) such as those commercially available from Gaf Corporation (Wayne, N.J., USA) under the GAFQUAT tradename (e.g., GAFQUAT 755N); cationic diallyl quaternary ammonium-containing polymers, including, for example, dimethyldiallylammonium chloride homopolymer and copolymers of acrylamide and dimethyldiallylammonium chloride, referred to in the industry (CTFA) as Polyquaternium 6 and Polyquaternium 7, respectively; and mineral acid salts of amino-alkyl esters of homo- and co-polymers of unsaturated carboxylic acids having from 3 to 5 carbon atoms, as described in U.S. Pat. No. 4,009,256.

Other cationic polymers that can be used include polysaccharide polymers, such as cationic cellulose derivatives and cationic starch derivatives.

Cationic polysaccharide polymer materials suitable for use herein include those of the formula:

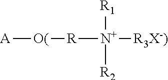

wherein: A is an anhydroglucose residual group, such as a starch or cellulose anhydroglucose residual, R is an alkylene oxyalkylene, polyoxyalkylene, or hydroxyalkylene group, or combination thereof, R₁, R₂, and R₃ independently are alkyl, aryl, alkylaryl, arylalkyl, alkoxyalkyl, or alkoxyaryl groups, each group containing up to about 18 carbon atoms, and the total number of carbon atoms for each cationic moiety (i.e., the sum of carbon atoms in R₁, R₂ and R₃) preferably being about 20 or less, and X is an anionic counterion, as previously described.

Cationic cellulose is available from Amerchol Corp. (Edison, N.J., USA) in their Polymer JR® and LR® series of polymers, as salts of hydroxyethyl cellulose reacted with trimethyl ammonium substituted epoxide, referred to in the industry (CTFA) as Polyquaternium 10. Another type of cationic cellulose includes the polymeric quaternary ammonium salts of hydroxyethyl cellulose reacted with lauryl dimethyl ammonium-substituted opoxide, referred to in the industry (CTFA) as Polyquaternium 24. These materials are available from Amerchol Corp. (Edison, N.J., USA) under the tradename Polymer LM-200®.

Other cationic polymers that can be used include cationic guar gum derivatives, such as guar hydroxypropyltrimonium chloride (commercially available from Celanese Corp. in their Jaguar R series). Other materials include quaternary nitrogen-containing cellulose ethers (e.g., as described in U.S. Pat. No. 3,962,418), and copolymers of etherified cellulose and starch (e.g., as described in U.S. Pat. No. 3,958,581).

As discussed above, the cationic polymer hereof is water soluble. This does not mean, however, that it must be soluble in the composition. Preferably however, the cationic polymer is either soluble in the composition, or in a complex coacervate phase in the composition formed by the cationic polymer and anionic material. Complex coacervates of the cationic polymer can be formed with anionic surfactants or with anionic polymers that can optionally be added to the compositions hereof (e.g., sodium polystyrene sulfonate). However, the present composition is substantially free of anionic surfactants. Where anionic surfactants are present, they are used only in amounts of less than about 5%, preferably less than about 3% and most preferably less than about 2% by weight of the composition.

The compositions may comprise at from about 0.05% to about 10% of the additional cationic conditioning polymer by weight of the composition. In one embodiment, the compositions comprise from about 0.05% to about 2%, by weight of the composition, of the cationic conditioning polymer.

### Aqueous Carrier

The compositions also comprise an aqueous carrier. Preferably, the aqueous carrier is present in an amount of from about 50% to about 99.8% by weight of the composition. The aqueous carrier comprises a water phase which can optionally include other liquid, water-miscible or water-soluble solvents such as lower alkyl alcohols, e.g. C₁-C₅ alkyl monohydric alcohols, preferably C₂-C₃ alkyl alcohols. However, the liquid fatty alcohol must be miscible in the aqueous phase of the composition. Said fatty alcohol can be naturally miscible in the aqueous phase or can be made miscible through the use of cosolvents or surfactants.

In one embodiment, the composition is an emulsion, having viscosity at 25° C. of at least about 5,000 cP preferably from about 8,000 cP to about 50,000 cP, more preferably from about 15,000 cP to about 35,000 cP. Viscosity is determined by a Brookfield RVT, at 20 RPM.

### Anti-Dandruff Actives

The compositions may also comprise an anti-dandruff active. Suitable non-limiting examples of anti-dandruff actives include pyridinethione salts (ie. zinc pyrithione), azoles, selenium sulfide, particulate sulfur, keratolytic agents, and mixtures thereof. Such anti-dandruff actives should be physically and chemically compatible with the essential components of the composition, and should not otherwise unduly impair product stability, aesthetics or performance.

Pyridinethione anti-microbial and anti-dandruff agents are described, for example, in U.S. Pat. No. 2,809,971; U.S. Pat. No. 3,236,733; U.S. Pat. No. 3,753,196; U.S. Pat. No. 3,761,418; U.S. Pat. No. 4,345,080; U.S. Pat. No. 4,323,683; U.S. Pat. No. 4,379,753; and U.S. Pat. No. 4,470,982.

Azole anti-microbials include imidazoles such as climbazole and ketoconazole.

Selenium sulfide compounds are described, for example, in U.S. Pat. No. 2,694,668; U.S. Pat. No. 3,152,046; U.S. Pat. No. 4,089,945; and U.S. Pat. No. 4,885,107.

Sulfur may also be used as a particulate anti-microbial/anti-dandruff agent in the compositions.

The compositions may further comprise one or more keratolytic agents such as salicylic acid.

Additional anti-microbial actives may include extracts of melaleuca (tea tree) and charcoal.

### Particles

The compositions may also comprise particles. Useful particles can be natural, inorganic, synthetic, or semi-synthetic. In the present invention, it is preferable to incorporate no more than about 20%, more preferably no more than about 10% and even more preferably no more than 2%, by weight of the composition, of particles. In one embodiment, the particles have an average mean particle size of less than about 300 μm.

Non-limiting examples of natural particles comprise hydrophobic tapioca starch, corn starch and dried fruit particles.

Non-limiting examples of inorganic particles include colloidal silicas, fumed silicas, precipitated silicas, silica gels, magnesium silicate, glass particles, talcs, micas, sericites, and various natural and synthetic clays including bentonites, hectorites, and montmorillonites.

Examples of synthetic particles comprise silicone resins, poly(meth)acrylates, polyethylene, polyester, polypropylene, polystyrene, polyurethane, polyamide (e.g., Nylon®), epoxy resins, urea resins, acrylic powders, and the like.

Non-limiting examples of hybrid particles include sericite & crosslinked polystyrene hybrid powder, and mica and silica hybrid powder.

### Other Ingredients

The compositions herein can contain a variety of other optional components suitable for rendering such compositions more cosmetically or aesthetically acceptable or to provide them with additional usage benefits. Such conventional optional ingredients are well-known to those skilled in the art.

A wide variety of additional ingredients can be formulated into the present composition. These include: other conditioning agents; hair-hold polymers; detersive surfactants such as nonionic, amphoteric, and zwitterionic surfactants; additional thickening agents and suspending agents such as xanthan gum, guar gum, hydroxyethyl cellulose, methyl cellulose, hydroxyethylcellulose, starch and starch derivatives; viscosity modifiers such as methanolamides of long chain fatty acids such as cocomonoethanol amide; crystalline suspending agents; pearlescent aids such as ethylene glycol distearate; preservatives such as benzyl alcohol, methyl paraben, propyl paraben and imidazolidinyl urea; polyvinyl alcohol; ethyl alcohol; pH adjusting agents, such as citric acid, sodium citrate, succinic acid, phosphoric acid, sodium hydroxide, sodium carbonate; salts, in general, such as potassium acetate and sodium chloride; coloring agents, such as any of the FD&C or D&C dyes; hair oxidizing (bleaching) agents, such as hydrogen peroxide, perborate and persulfate salts; hair reducing agents, such as the thioglycolates; perfumes; sequestering agents, such as disodium ethylenediamine tetra-acetate; and polymer plasticizing agents, such as glycerin, disobutyl adipate, butyl stearate, and propylene glycol. Such optional ingredients generally are used individually at levels from about 0.01% to about 10.0%, preferably from about 0.05% to about 5.0% by weight of the composition.

### Method of Use

A method of straightening hair may include administrating an effective amount of the composition herein to hair. The composition includes at least one emulsifying silicone elastomer, at least one naturally derived deposition polymer, at least one silicone conditioning agent, and an aqueous carrier. Each of these components is discussed, in detail, hereinbefore.

The method of using the composition herein may include the steps of shampooing, conditioning, then drying hair. Once the hair is dry, an effective amount of the composition is applied to hair. Preferably, the composition is applied throughout the hair, sequentially, in sections. The hair may then by styled as the composition dries, optionally with the assistance of a blow dryer. Notably, when using a blow drier, no heat is required for hair to straighten. Therefore, the blow drier may be adjusted to apply air without heat. While the composition results in a straightening effect without the application of heat to hair, a user may optionally apply a heated styling device, such as a flat iron, to achieve a desired style without diminishing the straightening effect of the composition herein.

The straightening effect of the composition herein has a cumulative effect. Therefore, the method for using the composition should be repeated, once a day, over a series of consecutive days. Specifically, the method should be repeated over a period of at least 3 days, and preferably over a series of 5 consecutive days.

The composition herein is applied as a “leave-in” conditioner. Leave-in conditioners are compositions designed to be applied to hair without rinsing. The hair straightening effect achieved by the composition is diminished when rinsed. Therefore, according to the method herein, the composition is not rinsed from the hair after application.

### Examples

All parts, percentages, and ratios herein are by weight unless otherwise specified. Some components may come from suppliers as dilute solutions. The levels given reflect the weight percent of the active material, unless otherwise specified.

| INGREDIENT | Wt. % | | |
|---|---|---|---|
| Hydroxypropyl Methycellulose | 0.6 | .08 | 0.1 |
| Polyquaternium-10¹ | 0.6 | — | 0.1 |
| Tapioca Starch² | 0.1 | 0.2 | 0.1 |
| Maltodextrin/Aloe Barbadenisis Leaf Juice³ | 0.5 | 0.2 | — |
| Hydroxypropyltrimonium Hydrolyzed Maize Starch⁴ | 0.75 | 0.8 | 0.9 |
| Babassuamidopropyltrimonium | 1 | 1.3 | 1.1 |
| Methosulfate/Behenamidopropyltrimonium | | | |
| Methosulfate/Stearyl Alcohol | | | |
| Behentrimonium Methosulfate (and) Cetearyl | 2 | 1 | 3 |
| Alcohol⁵ | | | |
| Glyceryl Stearate/PEG-100 Stearate⁶ | 1 | 1.5 | 1 |
| Cetyl Dimethicone | 2 | 1.5 | 1 |
| Calophyllum Inophyllum Seed Oil | 0.5 | 1 | 0.75 |
| Dimethicone⁷ | 1 | 0.8 | 1.5 |
| Dimethicone⁸ | 3 | 3.5 | 3 |
| Dimethicone/Dimethicone/PEG-10/15 Crosspolymer | 5.5 | 6 | 4 |
| Caprylyl Glycol⁹ | 0.15 | 0.2 | 0.1 |
| Water/Hydrolyzed Wheat Protein PG-Propyl | 0.1 | 0.15 | 0.1 |
| Silanetriol¹⁰ | | | |
| Phenoxyethanol | 0.7 | 0.5 | 0.9 |
| Fragrance (Parfum) | 0.3 | 0.5 | 0.4 |
| Water | q.s. | q.s. | q.s. |

¹UCare Polymer JR30M, MW = 2.0 MM, charge density = 1.32 meq./gram, from Dow Chemicals
²Tapioca Pure from AkzoNobel
³CoVera ™ Dry from Hallstar
⁴MiruStyle ™ MFP PE from Croda
⁵Incroquat ™ from Croda
⁶Lipomulse ® 165 from Lipo Chemicals
⁷Xiameter PMX200 Silicone 100,000 cs from Dow Corning
⁸Xiameter PMX-200 Silicone FL 5.0 cs From Dow Corning
⁹KSG210 ® from Shin Etsu
¹⁰Crodasone ™ W from Croda

It is understood that the examples and embodiments described herein are for illustrative purposes only and that various modifications or changes in light thereof will be suggested to one skilled in the art without departing from the scope of the present invention.

What is claimed is:

1. A method for straightening hair comprising applying an effective amount of a hair straightening composition to hair, said composition comprising at least one emulsifying silicone elastomer, at least one naturally derived deposition polymer; at least one silicone conditioning agent, and an aqueous carrier, wherein said composition is not rinsed from the hair.

2. A method according to claim 1, wherein a straightening effect is achieved without the application of heat.

3. A method according to claim 1, wherein a straightening effect is achieved without damaging the hair.

4. A method according to claim 1, wherein a cumulative straightening effect is achieved by repeating the method of claim 1 for at least 3 consecutive days.

5. A method according to claim 1, wherein a cumulative straightening effect is achieved by repeating the method of claim 1 for at least 5 consecutive days.

6. A method according to claim 1, wherein the emulsifying silicone elastomer is selected from polyoxyalkylenated silicone elastomers and polyglycerolated silicone elastomers.

7. A method according to claim 1, wherein said emulsifying silicone elastomer is present at from about 2% to about 15% by weight of the composition.

8. A method according to claim 1, wherein said naturally derived deposition polymer is selected from cellulose deposition polymers, guar cationic deposition polymers, and non-guar galactomannan deposition polymers.

9. A method according to claim 1, wherein said naturally derived deposition polymer is a cationically modified starch polymer.

10. A method according to claim 9, wherein the source of said cationically modified starch polymer is selected from the group consisting of corn starch, wheat starch, rice starch, waxy corn starch, oat starch, cassia starch, waxy barley, waxy rice starch, glutenous rice starch, sweet rice starch, amioca, potato starch, tapioca starch, oat starch, sago starch, sweet rice, and mixtures thereof.

11. A method according to claim 1, wherein said composition is substantially free of anionic surfactants.

12. A method according to claim 1, wherein said composition is substantially free of reactive hair straightening chemicals.

13. A method according to claim 12, wherein said reactive hair straightening chemicals are selected from the group consisting of thiogycolic acid, ammonium thioglycolate, cysteamine, sodium hydroxide, calcium hydroxide, guanidine hydroxide, and mixtures thereof.

14. A method according to claim 12, wherein said composition is substantially free of formaldehyde, formol, mercaptans, sulfonic acids, sulfites, and mixtures thereof.
